(19) United States
(12) Patent Application Publication
(10) Pub. No.: US 2024/0182992 A1
(43) Pub. Date: Jun. 6, 2024

(54) METHODS AND COMPOSITIONS RELATED TO COOPERATIVE PRIMERS AND REVERSE TRANSCRIPTION

(71) Applicant: CO-DIAGNOSTICS, INC., Salt Lake City, UT (US)

(72) Inventor: Kenneth K.C. BRAMWELL, Bountiful, UT (US)

(21) Appl. No.: 18/566,302

(22) PCT Filed: Jun. 2, 2022
(86) PCT No.: PCT/US2022/031913

§ 371 Date: Dec. 1, 2023

Related U.S. Application Data
(60) Provisional application No. 63/196,004, filed on Jun. 2, 2021.

(51) Int. Cl. C12Q 1/70; C12Q 1/6844

(52) U.S. Cl. CPC C12Q 1/701; C12Q 1/6844

(57) ABSTRACT

Disclosed are compositions, methods, and kits relating to amplifying and detecting RNA using Co-Primers and conditionally active reverse transcription primers. The conditionally active reverse transcriptase primers work in concert with dual Co-Primer assays to improve sensitivity toward RNA targets without detracting from the improved amplification specificity of the associated Co-Primers.

### CROSS-REFERENCE TO RELATED APPLICATIONS

This application claims benefit of U.S. Provisional Application No. 63/196,004, filed Jun. 2, 2021, incorporated herein by reference in its entirety.

### BACKGROUND OF THE INVENTION

Detection of nucleic acid signatures through amplification via Polymerase Chain Reaction (PCR) is the foundation of many modern molecular diagnostic assays. Oligonucleotide (oligo) primer pairs are designed to bind to unique sequence signatures in the desired target and to thereby prime the enzymatic activity of thermostable DNA Polymerase, followed by heat denaturation. Multiple cycles of target binding, priming, polymerization, and denaturation lead to the generation of millions or billions of copies of the target amplicon, which are readily detected by various means. Although this combination of molecular reagents can be exquisitely sensitive and specific under the right conditions, it can also be notoriously promiscuous and indiscriminate under the wrong conditions.

Cooperative Primers (CoPrimers) were developed to mitigate the risk of undesirable primer-dimer formation (U.S. Pat. No. 10,093,966, hereby incorporated by reference in its entirety for its disclosure concerning CoPrimers). CoPrimers use an inert, flexible linker to combine two short oligonucleotides into a single molecule. These two oligo sequences (Primer and Capture/Probe) are designed to recognize adjacent sequences on a common target but are short enough that neither oligo can consistently bind alone under PCR reaction conditions. However, the spatial constraint provided by the linker molecule allows the two linked oligos to cooperatively stabilize each oligo binding event on an appropriate target sequence. In one commonly used CoPrimer configuration, the 3′ end of the capture oligo is conjugated directly to the flexible linker molecule and is therefore unavailable to DNA Polymerase for priming and extension. The 3′ end of the Primer oligo is available for priming but is short, and therefore does not bind its solitary target sequence with high efficiency (as would exist in a primer-dimer lacking the capture sequence). In a commonly used CoPrimer configuration where the Capture/Probe sequence also contains a fluorescent label coupled with one or more quencher molecules, signal generation is conditional upon cooperative binding to a target sequence, priming, and the 5′->3′ exonuclease activity of the DNA polymerase acting upon the bound capture sequence to degrade it and thereby generate fluorescence. If a primer-dimer involving the Primer sequence alone does manage to form and efficiently propagate, it contains no binding site for the Capture/Probe sequence and such a molecule would thereby competitively suppresses specific exonuclease-based fluorescence.

The short length of oligos used in CoPrimers make each of them especially sensitive to destabilization by mismatches, which further enhances their target specificity. Bypass amplification is another serious concern in traditional PCR assays, whereby a mismatched natural target is bound and primed at a low frequency but then incorporates the full artificial primer sequence into the resulting molecule, forming a perfect-match target for future rounds of amplification and effectively curing the defect that is relied upon to provide assay specificity. Because CoPrimer capture/probe sequences are not incorporated into the resulting amplicon, any mismatches present that portion of the sequence cannot be cured in this way and mismatches will persist for specificity interrogation in every cycle of PCR.

The unique features of CoPrimers provide much higher specificity through molecular suppression of primer-dimer formation, through competitive suppression of Probe fluorescence should primer-dimers form, and through resistance to bypass amplification. The net reduction in primer-dimer formation alone was reported to be a 2.5 million-fold improvement. However, the CoPrimer molecule is more complex than a regular primer and it is reasonable to assume that cooperative binding may involve more complex kinetics than a comparable regular primer. Any theoretical inefficiencies that could occur due to complex binding kinetics could manifest as slightly lower amplification efficiency, but this is easily solved by additional cycling. Traditional PCR assays often include a Ct cutoff level beyond which non-specific amplification produces a false amplification signal that must be ignored. This prevents additional cycling, and often comes at a cost with diminished assay sensitivity where late amplifying True Positives also must be ignored due to the specificity requirement imposed at the Ct cutoff level. The enhanced specificity of CoPrimer PCR assays allow additional cycling to occur, thereby retaining full sensitivity for True Positives that may amplify late or with lower efficiency.

The nucleic acid signatures for some natural targets are formed by RNA, and prior to PCR amplification this RNA must first be converted to a DNA molecule through a Reverse Transcriptase (RT) step. The RT step occurs once, and any inefficiency in this step cannot be recovered later through additional PCR cycling. This RT step also requires priming, and One-Step RT-PCR often requires use of target-specific primers that will not later produce off-target priming in the PCR reaction. Many diagnostic assays use the same primer for both RT and for PCR, since it is already designed to be assay specific, and will typically function under both conditions.

CoPrimers can be used successfully for RT priming, and any theoretical concern about inefficiency at this step could be addressed by substituting a regular Reverse primer for one of the CoPrimers in each assay. However, any theoretical gain in sensitivity would detract from the added specificity provided by a dual CoPrimer assay. A regular primer could be added downstream of the dual CoPrimer assay to help bolster the RT efficiency, but this primer might then independently participate in the PCR reaction, again detracting from the specificity gained by the reverse CoPrimer.

What is needed in the art are empirically selected, conditionally active oligonucleotide molecules (RT Primers) that work in concert with dual CoPrimer assays to improve sensitivity toward RNA targets without detracting from the improved amplification specificity of the associated CoPrimers.

### BRIEF SUMMARY OF THE INVENTION

Disclosed herein is a method of transcribing RNA into cDNA, the method comprising: providing a target RNA sequence; providing a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows both the said first and second nucleic acid sequences to hybridize to the target RNA at the same time; providing a conditionally active reverse transcriptase primer, wherein said conditionally active reverse transcriptase primer has a melting temperature between about 45° C. and about 55° C.; providing reverse transcriptase; and providing conditions that allow for transcription of the RNA target into cDNA; thereby transcribing RNA into cDNA.

Also disclosed is a kit comprising: a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows both the said first and second nucleic acid sequences to hybridize to the target RNA at the same time; and a conditionally active reverse transcriptase primer, wherein said conditionally active reverse transcriptase primer has a melting temperature between about 45° C. and about 55° C.

Further disclosed is a method of detecting RNA, the method comprising: providing a target RNA sequence; providing a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows both the said first and second nucleic acid sequences to hybridize to the target RNA at the same time; providing a conditionally active reverse transcriptase primer, wherein said conditionally active reverse transcriptase primer has a melting temperature between about 45° C. and about 55° C.; providing reverse transcriptase; providing conditions that allow for transcription of the RNA target into cDNA, thereby producing cDNA; amplifying the cDNA product; and detecting the amplified cDNA produced.

### BRIEF DESCRIPTION OF THE DRAWINGS

FIG. 1 shows a run profile with reverse transcription and PCR occurring at distinct temperatures.

FIG. 2 shows staggered RT primer oligo design which allows empirical PCR competency evaluation. These are represented by SEQ ID NOS: 1-4, respectively.

FIG. 3A-B shows empirical PCR competency evaluation. A—Dual-CoPrimer assay amplification with RP7(red) or RP8(blue) added. B—Forward CoPrimer only, with RP7(red) or RP8(blue). RP7 provides inefficient amplification with delayed Cq, RP8 is PCR incompetent (no amplification).

FIG. 4A-H shows reverse transcriptase primer improves co-primer assay sensitivity for an RNA target.

FIG. 5 shows reverse transcriptase primers retain specificity in NTC reactions under exacerbated experimental conditions.

### DETAILED DESCRIPTION OF THE INVENTION

The disclosed method makes use of certain materials and procedures which allow amplification of nucleic acid sequences and whole genomes or other highly complex nucleic acid samples. These materials and procedures are described in detail below.

### Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only and is not intended to be limiting.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine, hypoxathanine, isocytosine, isoguanine, etc. It may include modified bases, including locked nucleic acids, peptide nucleic acids and others known to those skilled in the art.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “peptide nucleic acid” (PNA) is a polymer comprising two or more peptide nucleic acid monomers. The polymer can additionally comprise elements such as labels, quenchers, blocking groups, or the like. The monomers of the PNA can be unsubstituted, unmodified, substituted or modified.

By “cooperative nucleic acid” is meant a nucleic acid sequence which incorporates minimally a first nucleic acid sequence and a second nucleic acid sequence, wherein the second nucleic acid sequence hybridizes to the target nucleic acid downstream of the 3′ end of the first nucleic acid sequence. The 3′ end of the nucleic acid can be extendable, as discussed elsewhere herein. In one example, the first nucleic acid is a primer, and the second nucleic acid is a capture sequence. The first and second nucleic acid sequences can be separated by a linker, for example.

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified. By “cooperative primer,” or first nucleic acid sequence, is meant a primer attached via a linker to a second nucleic acid sequence, also referred to as a capture sequence. The second nucleic acid sequence, or capture sequence, can hybridize to the template nucleic acid downstream of the 3′ end of the primer, or first nucleic acid sequence. By “normal primer” is meant a primer which does not have a capture sequence, or second nucleic acid sequence, attached to it via a linker.

By “capture sequence,” which is also referred to herein as a “second nucleic acid sequence” is meant a sequence which hybridizes to the target nucleic acid and allows the first nucleic acid sequence, or primer sequence, to be in close proximity to the target region of the target nucleic acid.

By “amplicon” is meant a polynucleotide generated during the amplification of a polynucleotide of interest. In one example, an amplicon is generated during a polymerase chain reaction.

By “amplification rate modifiers” is meant an agent capable of affecting the rate of polymerase extension.

As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA.

By “base substitution” is meant a substituent of a nucleobase polymer that does not cause significant disruption of the hybridization between complementary nucleotide strands.

“Downstream” is relative to the action of the polymerase during nucleic acid synthesis or extension. For example, when the Taq polymerase extends a primer, it adds bases to the 3′ end of the primer and will move towards a sequence that is “downstream from the 3′ end of the primer.”

A “target region” is a region of a target nucleic acid that is to be amplified, detected or both.

The “Tm” (melting temperature) of a nucleic acid duplex under specified conditions is the temperature at which half of the nucleic acid sequences are disassociated and half are associated. As used herein, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

The term “linker” means the composition joining the first and second nucleic acids to each other. The linker comprises at least one non-extendable moiety, but may also comprise extendable nucleic acids, and can be any length. The linker may be connected to the 3′ end, the 5′ end, or can be connected one or more bases from the end (“the middle”) of both the first and second nucleic acid sequences. The connection can be covalent, hydrogen bonding, ionic interactions, hydrophobic interactions, and the like. The term “non-extendable” has reference to the inability of the native Taq polymerase to recognize a moiety and thereby continue nucleic acid synthesis. A variety of natural and modified nucleic acid bases are recognized by the polymerase and are “extendable.” Examples of non-extendable moieties include among others, fluorophores, quenchers, polyethylene glycol, polypropylene glycol, polyethylene, polypropylene, polyamides, polyesters and others known to those skilled in the art. In some cases, even a nucleic acid base with reverse orientation (e.g. 5′ ACGT 3′ 3′A 5′ 5′ AAGT 3′) or otherwise rendered such that the Taq polymerase could not extend through it could be considered “non-extendable.”The term “non-nucleic acid linker” as used herein refers to a reactive chemical group that is capable of covalently attaching a first nucleic acid to a second nucleic acid, or more specifically, the primer to the capture sequence. Suitable flexible linkers are typically linear molecules in a chain of at least one or two atoms, more typically an organic polymer chain of 1 to 12 carbon atoms (and/or other backbone atoms) in length. Exemplary flexible linkers include polyethylene glycol, polypropylene glycol, polyethylene, polypropylene, polyamides, polyesters and the like.

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 90% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

As used herein, “amplify, amplifying, amplifies, amplified, amplification” refers to the creation of one or more identical or complementary copies of the target DNA. The copies may be single stranded or double stranded. Amplification can be part of a number of processes such as extension of a primer, reverse transcription, polymerase chain reaction, nucleic acid sequencing, rolling circle amplification and the like.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid.

“Detect” refers to identifying the presence, absence, or amount of the analyte to be detected.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides. In one embodiment, the fragment comprises at least about 50, 75, 80, 85, 89, 90, or 100 nucleotides of an Ebola polynucleotide or other RNA viral polynucleotide.

As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

“Reverse transcriptase” (RT) is an enzyme used to generate complementary DNA (cDNA) from an RNA template, a process termed reverse transcription. Reverse transcriptase is commonly used in research to apply the polymerase chain reaction technique to RNA in a technique called reverse transcription polymerase chain reaction (RT-PCR). The classical PCR technique can be applied only to DNA strands, but, with the help of reverse transcriptase, RNA can be transcribed into DNA, thus making PCR analysis of RNA molecules possible. Reverse transcriptase is used also to create cDNA libraries from mRNA.

A variety of additional terms are defined or otherwise characterized herein.

### Compositions and Methods

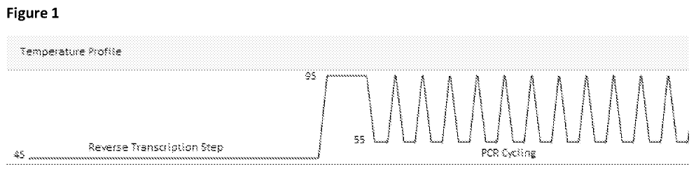

Disclosed herein are empirically selected, conditionally active oligonucleotide molecules (RT Primers) that work in concert with dual CoPrimer assays to improve sensitivity toward RNA targets without detracting from the improved amplification specificity of the associated CoPrimers. To achieve this, the sequence 50 to 200 bp downstream of a dual CoPrimer assay was evaluated, and oligonucleotide primers were designed with predicted melting temperatures between 45° C. (the hold temperature of the Reverse Transcriptase step) and 55° C. (the lowest temperature condition during PCR cycling for the CoPrimer assay) (FIG. 1).

The present invention relates to the use of reverse transcription with cooperative nucleic acids, such as primers and probes. Disclosed herein is a method of transcribing RNA into cDNA, the method comprising: providing a target RNA sequence; providing a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows (or requires) both the said first and second nucleic acid sequences to hybridize to the target RNA at the same time; providing a conditionally active reverse transcriptase primer, wherein said conditionally active reverse transcriptase primer has a melting temperature between about 45° C. and about 55° C.; providing reverse transcriptase; and providing conditions that allow for transcription of the RNA target into cDNA; thereby transcribing RNA into cDNA.

The conditionally active reverse transcriptase primer can have a melting temperature of about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70° C., or any amount above, below, or in between. In a preferred embodiment, the melting temperature is between 40° C. and 60° C. In another preferred embodiment, the melting temperature is between 45° C. and 55° C. The hold temperature of the Reverse Transcriptase step is typically about 45° C., and 55° C. is approximately the lowest temperature condition during PCR cycling (FIG. 1).

The conditionally active reverse transcriptase primer can bind the RNA target at a distance of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, or more nucleotides from where the co-primer binds the RNA target. In a preferred embodiment, the conditionally active reverse transcriptase primer can bind the RNA target at a distance of at least 2-30 nucleotides from where the co-primer binds the RNA target. In yet another preferred embodiment, the conditionally active reverse transcriptase primer can bind the RNA target at a distance of at least 5 nucleotides from where the co-primer binds the RNA target. In yet another preferred embodiment, the conditionally active reverse transcriptase primer can bind the RNA target at a distance of at least 20 nucleotides from where the co-primer binds the RNA target. In yet another preferred embodiment, the conditionally active reverse transcriptase primer can bind the RNA target at a distance of at least 50, but not more than 300, nucleotides from where the co-primer binds the RNA target. In yet another preferred embodiment, the conditionally active reverse transcriptase primer can bind the RNA target at a distance of at least 10, but not more than 200, nucleotides from where the co-primer binds the RNA target.

In one embodiment, the first nucleic acid molecule of the co-primer will not hybridize to the target without the second nucleic acid molecule hybridizing to the target. In another embodiment, the second nucleic acid molecule of the co-primer will not hybridize to the target without the first nucleic acid molecule hybridizing to the target. In yet another embodiment, neither the first nor the second nucleic acid molecule of the co-primer will hybridize to the target without the other hybridizing to the target.

Also disclosed herein is a method of detecting RNA, the method comprising: providing a target RNA sequence; providing a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows both the said first and second nucleic acid sequences to hybridize to the target RNA at the same time; providing a conditionally active reverse transcriptase primer, wherein said conditionally active reverse transcriptase primer has a melting temperature between about 45° C. and about 55° C.; providing reverse transcriptase; providing conditions that allow for transcription of the RNA target into cDNA, thereby producing cDNA; amplifying the cDNA product; and detecting the amplified cDNA produced.

The method disclosed above is useful in detecting RNA. The source, identity, and preparation of many such RNA nucleic acid samples are known. It is preferred that nucleic acid samples known or identified for use in amplification or detection methods be used for the method described herein. Whether translated into protein or functioning in a regulatory manner, RNAs underlie many aspects of cellular function, and it has become clear that their dysregulation is associated with various diseases. Understanding RNA biology requires detection, quantification, and sequence-specific characterization of the composite RNAs.

RNA amplification and detection can also be useful in detecting and quantifying RNA-based viruses. For example, the virus to be detected can be, but is not limited to, paramyxoviruses, hendra and nipah viruses; measles; severe acute respiratory syndrome coronavirus (SARS), SARS-CoV-2 (the disease that causes COVID-19); Middle east respiratory syndrome coronavirus (MERS); picornaviruses; poliomyelitis (‘Polio’); hepatitis A virus (HAV); rotavirus; human immunodeficiency virus (HIV); human T-cell lymphotropic virus (HTLV); hepatitis C virus (HCV;) hepatitis E virus (HEV); rabies; Ebola virus disease (EVD;) Marburg virus; Lassa fever; Lymphocytic choriomeningitis virus (LCMV); Arboviruses (‘ARthropod-BOrne viruses’); Japanese encephalitis (JE); West Nile fever; Yellow fever; Dengue fever; Zika virus; Equine encephalitis viruses; Chikungunya; O'nyong-nyong; Bunyaviruses; Rift valley fever and Crimean-Congo haemorrhagic fever; and Hantavirus.

The methods disclosed herein are useful in a variety of primer extension/amplification reactions known to those skilled in the art, including, but not limited to the polymerase chain reaction, rolling circle amplification, nucleic acid sequencing and others. The cooperative primers and probes of this invention can also be used in applications that have post extension/amplification steps, such as hybridization to an array. Because the cooperative primers/probes in this invention substantially reduce primer-dimers, they are of particular use in multiplexed and highly multiplexed reactions.

“RT-PCR” as used herein refers to the combination of reverse transcription and PCR in a single assay. “Reverse transcription” is a process whereby an RNA template is transcribed into a DNA molecule by a reverse transcriptase enzyme. Thus, “reverse transcriptase” describes a class of polymerases characterized as RNA-dependent DNA polymerases, that is, such polymerases use an RNA template to synthesize a DNA molecule. Historically, reverse transcriptases have been used to reverse-transcribe mRNA into cDNA. However, reverse transcriptases can be used to reverse-transcribe other types of RNAs such as viral genomic RNA or viral sub-genomic RNA. Standard reverse transcriptases include Maloney Murine Leukemia Virus Reverse Transcriptase (MoMuLV RT) and Avian myoblastosis virus (AMV). These enzymes have 5′->3′ RNA-dependent DNA polymerase activity, 5′->3′ DNA-dependent DNA polymerase activity, and RNase H activity. However, unlike many DNA-dependent DNA polymerases, these enzymes lack 3′->5′ exonuclease activity necessary for “proofreading,” (i.e., correcting errors made during transcription). After a DNA copy of an RNA has been prepared, the DNA copy may be subjected to various DNA amplification methods such as PCR.

A variety of amplification enzymes are well known in the art and include, for example, DNA polymerase, RNA polymerase, reverse transcriptase, Q-beta replicase, thermostable DNA and RNA polymerases. Because these and other amplification reactions are catalyzed by enzymes, in a single step assay that the nucleic acid releasing reagents and the detection reagents should not be potential inhibitors of amplification enzymes if the ultimate detection is to be amplification based.

Amplification of the nucleic acid molecules in a nucleic acid sample can result from replication of at least 0.01% of the nucleic acid sequences in the nucleic acid sample, at least 0.1% of the nucleic acid sequences in the nucleic acid sample, at least 1% of the nucleic acid sequences in the nucleic acid sample, at least 5% of the nucleic acid sequences in the nucleic acid sample, at least 10% of the nucleic acid sequences in the nucleic acid sample, at least 20% of the nucleic acid sequences in the nucleic acid sample, at least 30% of the nucleic acid sequences in the nucleic acid sample, at least 40% of the nucleic acid sequences in the nucleic acid sample, at least 50% of the nucleic acid sequences in the nucleic acid sample, at least 60% of the nucleic acid sequences in the nucleic acid sample, at least 70% of the nucleic acid sequences in the nucleic acid sample, at least 80% of the nucleic acid sequences in the nucleic acid sample, at least 90% of the nucleic acid sequences in the nucleic acid sample, at least 95% of the nucleic acid sequences in the nucleic acid sample, at least 96% of the nucleic acid sequences in the nucleic acid sample, at least 97% of the nucleic acid sequences in the nucleic acid sample, at least 98% of the nucleic acid sequences in the nucleic acid sample, or at least 99% of the nucleic acid sequences in the nucleic acid sample.

The various sequence representations described above and elsewhere herein can be, for example, for 1 target sequence, 2 target sequences, 3 target sequences, 4 target sequences, 5 target sequences, 6 target sequences, 7 target sequences, 8 target sequences, 9 target sequences, 10 target sequences, 11 target sequences, 12 target sequences, 13 target sequences, 14 target sequences, 15 target sequences, 16 target sequences, 17 target sequences, 18 target sequences, 19 target sequences, 20 target sequences, 25 target sequences, 30 target sequences, 40 target sequences, 50 target sequences, 75 target sequences, or 100 target sequences. The sequence representation can be, for example, for at least 1 target sequence, at least 2 target sequences, at least 3 target sequences, at least 4 target sequences, at least 5 target sequences, at least 6 target sequences, at least 7 target sequences, at least 8 target sequences, at least 9 target sequences, at least 10 target sequences, at least 11 target sequences, at least 12 target sequences, at least 13 target sequences, at least 14 target sequences, at least 15 target sequences, at least 16 target sequences, at least 17 target sequences, at least 18 target sequences, at least 19 target sequences, at least 20 target sequences, at least 25 target sequences, at least 30 target sequences, at least 40 target sequences, at least 50 target sequences, at least 75 target sequences, or at least 100 target sequences.

The sequence representation can be, for example, for 1 target sequence, 2 different target sequences, 3 different target sequences, 4 different target sequences, 5 different target sequences, 6 different target sequences, 7 different target sequences, 8 different target sequences, 9 different target sequences, 10 different target sequences, 11 different target sequences, 12 different target sequences, 13 different target sequences, 14 different target sequences, 15 different target sequences, 16 different target sequences, 17 different target sequences, 18 different target sequences, 19 different target sequences, 20 different target sequences, 25 different target sequences, 30 different target sequences, 40 different target sequences, 50 different target sequences, 75 different target sequences, or 100 different target sequences. The sequence representation can be, for example, for at least 1 target sequence, at least 2 different target sequences, at least 3 different target sequences, at least 4 different target sequences, at least 5 different target sequences, at least 6 different target sequences, at least 7 different target sequences, at least 8 different target sequences, at least 9 different target sequences, at least 10 different target sequences, at least 11 different target sequences, at least 12 different target sequences, at least 13 different target sequences, at least 14 different target sequences, at least 15 different target sequences, at least 16 different target sequences, at least 17 different target sequences, at least 18 different target sequences, at least 19 different target sequences, at least 20 different target sequences, at least 25 different target sequences, at least 30 different target sequences, at least 40 different target sequences, at least 50 different target sequences, at least 75 different target sequences, or at least 100 different target sequences.

### Cooperative Primers

The use of a cooperative nucleic acid can decrease the amount of primer-dimer present by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent compared to the amount of primer-dimer present when a normal primer (a non-cooperative nucleic acid) is used.

The cooperative nucleic acid may be linear or circularized.

By “extendable on the 3′ end” is meant that the first nucleic acid is free on this end to be amplified or extended. This is meant to include heat activatable primers such as those described by Lebedev et al, among other technologies. This can mean that amplification can proceed from the 3′ end of the second nucleic acid sequence through the first nucleic acid sequence.

The first nucleic acid sequence is a primer, and the second nucleic acid sequence is alternatively referred to as a “capture nucleic acid sequence.” Either the first or the second sequence may have a detectable label, or a third sequence may have a detectable label. The first and second nucleic acid sequences can be attached via a linker, which can be a non-nucleic acid sequence. In one example, the linker can attach the 5′ end of the first nucleic acid sequence to the 3′ end of the second nucleic acid sequence. Alternatively, the first nucleic acid sequence is inverted such that the 5′ end of the first nucleic acid sequence is attached to the 5′ end of the second nucleic acid sequence. In yet another example, the 5′ end of the second nucleic acid sequence can be linked to the first nucleic acid sequence in the middle of the sequence. It is noted that by “middle of the sequence” is meant that the linker is not joined to the first nucleic acid sequence at either the 5′ end or the 3′ end of the nucleic acid, but rather is attached to a nucleotide internal to the nucleotides on the 5′ and 3′ ends.

In one example, the cooperative nucleic acid comprises 75, 70, 65, 60, 55, 50, 45, 40, 35, 30, 25, 20, 15, 10, or less continuous nucleotides in the same orientation. In other words, this is the number of nucleotides that are part of a single, unbroken nucleic acid sequence and oriented in the same 5′ to 3′ direction, or the 3′ to 5′ direction. By way of example, if the linker is a nucleic acid sequence, it can include the linker, if the nucleotides in the linker are in the same orientation as either the first or second nucleic acid sequence to which it is directly connected.

The linker can be made of nucleic acids, non-nucleic acids, or some combination of both. If the linker is made of nucleic acids, it can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleotides in length, or any number in between. Types of linkers are discussed elsewhere herein. The linker can be any length and can be longer or shorter than the combined length of the first and second nucleic acid sequences, longer or shorter than just the first nucleic acid sequence, or longer or shorter than the second nucleic acid sequence.

Furthermore, there can be a space on the target nucleic acid where the first nucleic acid sequence and the second nucleic acid sequence hybridize. In other words, there are two distinct regions on the target nucleic acid, one which hybridizes with the first nucleic acid sequence, and the other which hybridizes to the second nucleic acid sequence. The distance between the first and second regions on the target can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleotides in length.

Methods of amplification are disclosed elsewhere herein. More than one cooperative nucleic acid molecule can be provided, and they can have the same or different sequences. For example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 or more nucleic acid molecules of different sequences can be provided.

### Primer Design

In some embodiments, the isolated melting temperature “Tm” of the primer, also referred to herein as the first nucleic acid sequence, is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees below the reaction temperature used during the annealing phase, of PCR, or the extension phase of reactions with no annealing phase. Therefore, the melting temperature of the primer sequence can be between about 1° C. and 40° C., between about 3° C. and 20° C., between about 5° C. and 15° C. below the reaction temperature used in the PCR reaction. In a preferred embodiment, the isolated Tm is between about 7° C. and 12° C. below the reaction temperature. This provides for less than 50%, and more preferably less than 20% of the template to be hybridized to an isolated primer.

One of skill in the art can design primers with a given melting temperature based on many factors, such as length, and with increasing GC content. A simple formula for calculation of the (Tm) is:

Tm=4(G+C)+2(A+T)° C.

Furthermore, one of skill in the art will appreciate that the actual Tm is influenced by the concentration of Mg²⁺, K⁺, and cosolvents. There are numerous computer programs to assist in primer design.

To achieve the desired melting temperatures, the first nucleic acid sequence, or the primer, can be 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, and 40 bases in length. For example, the primers can be between about 5 and 26, between about 7 and 22, between about 9 and 17 bases in length depending on GC content.

Any desired number of primers of different nucleotide sequence can be used, but use of one or a few primers is preferred. The amplification reaction can be performed with, for example, one, two, three, four, five, six, seven, eight, nine, ten, eleven, twelve, thirteen, fourteen, fifteen, sixteen, or seventeen primers. More primers can be used. There is no fundamental upper limit to the number of primers that can be used. However, the use of fewer primers is preferred. When multiple primers are used, the primers should each have a different specific nucleotide sequence.

The amplification reaction can be performed with a single primer and, for example, with no additional primers, with 1 additional primer, with 2 additional primers, with 3 additional primers, with 4 additional primers, with 5 additional primers, with 6 additional primers, with 7 additional primers, with 8 additional primers, with 9 additional primers, with 10 additional primers, with 11 additional primers, with 12 additional primers, with 13 additional primers, with 14 additional primers, with 15 additional primers, with 16 additional primers, with 17 additional primers, with 18 additional primers, with 19 additional primers, with 20 additional primers, with 21 additional primers, with 22 additional primers, with 23 additional primers, with 24 additional primers, with 25 additional primers, with 26 additional primers, with 27 additional primers, with 28 additional primers, with 29 additional primers, with 30 additional primers, with 31 additional primers, with 32 additional primers, with 33 additional primers, with 34 additional primers, with 35 additional primers, with 36 additional primers, with 37 additional primers, with 38 additional primers, with 39 additional primers, with 40 additional primers, with 41 additional primers, with 42 additional primers, with 43 additional primers, with 44 additional primers, with 45 additional primers, with 46 additional primers, with 47 additional primers, with 48 additional primers, with 49 additional primers, with 50 additional primers, with 51 additional primers, with 52 additional primers, with 53 additional primers, with 54 additional primers, with 55 additional primers, with 56 additional primers, with 57 additional primers, with 58 additional primers, with 59 additional primers, with 60 additional primers, with 61 additional primers, with 62 additional primers, with 63 additional primers, with 64 additional primers, with 65 additional primers, with 66 additional primers, with 67 additional primers, with 68 additional primers, with 69 additional primers, with 70 additional primers, with 71 additional primers, with 72 additional primers, with 73 additional primers, with 74 additional primers, with 75 additional primers, with 76 additional primers, with 77 additional primers, with 78 additional primers, with 79 additional primers, with 80 additional primers, with 81 additional primers, with 82 additional primers, with 83 additional primers, with 84 additional primers, with 85 additional primers, with 86 additional primers, with 87 additional primers, with 88 additional primers, with 89 additional primers, with 90 additional primers, with 91 additional primers, with 92 additional primers, with 93 additional primers, with 94 additional primers, with 95 additional primers, with 96 additional primers, with 97 additional primers, with 98 additional primers, with 99 additional primers, with 100 additional primers, with 110 additional primers, with 120 additional primers, with 130 additional primers, with 140 additional primers, with 150 additional primers, with 160 additional primers, with 170 additional primers, with 180 additional primers, with 190 additional primers, with 200 additional primers, with 210 additional primers, with 220 additional primers, with 230 additional primers, with 240 additional primers, with 250 additional primers, with 260 additional primers, with 270 additional primers, with 280 additional primers, with 290 additional primers, with 300 additional primers, with 310 additional primers, with 320 additional primers, with 330 additional primers, with 340 additional primers, with 350 additional primers, with 360 additional primers, with 370 additional primers, with 380 additional primers, with 390 additional primers, with 400 additional primers, with 410 additional primers, with 420 additional primers, with 430 additional primers, with 440 additional primers, with 450 additional primers, with 460 additional primers, with 470 additional primers, with 480 additional primers, with 490 additional primers, with 500 additional primers, with 550 additional primers, with 600 additional primers, with 650 additional primers, with 700 additional primers, with 750 additional primers, with 800 additional primers, with 850 additional primers, with 900 additional primers, with 950 additional primers, with 1,000 additional primers, with 1,100 additional primers, with 1,200 additional primers, with 1,300 additional primers, with 1,400 additional primers, with 1,500 additional primers, with 1,600 additional primers, with 1,700 additional primers, with 1,800 additional primers, with 1,900 additional primers, with 2,000 additional primers, with 2,100 additional primers, with 2,200 additional primers, with 2,300 additional primers, with 2,400 additional primers, with 2,500 additional primers, with 2,600 additional primers, with 2,700 additional primers, with 2,800 additional primers, with 2,900 additional primers, with 3,000 additional primers, with 3,500 additional primers, or with 4,000 additional primers.

The amplification reaction can be performed with a single primer and, for example, with no additional primers, with fewer than 2 additional primers, with fewer than 3 additional primers, with fewer than 4 additional primers, with fewer than 5 additional primers, with fewer than 6 additional primers, with fewer than 7 additional primers, with fewer than 8 additional primers, with fewer than 9 additional primers, with fewer than 10 additional primers, with fewer than 11 additional primers, with fewer than 12 additional primers, with fewer than 13 additional primers, with fewer than 14 additional primers, with fewer than 15 additional primers, with fewer than 16 additional primers, with fewer than 17 additional primers, with fewer than 18 additional primers, with fewer than 19 additional primers, with fewer than 20 additional primers, with fewer than 21 additional primers, with fewer than 22 additional primers, with fewer than 23 additional primers, with fewer than 24 additional primers, with fewer than 25 additional primers, with fewer than 26 additional primers, with fewer than 27 additional primers, with fewer than 28 additional primers, with fewer than 29 additional primers, with fewer than 30 additional primers, with fewer than 31 additional primers, with fewer than 32 additional primers, with fewer than 33 additional primers, with fewer than 34 additional primers, with fewer than 35 additional primers, with fewer than 36 additional primers, with fewer than 37 additional primers, with fewer than 38 additional primers, with fewer than 39 additional primers, with fewer than 40 additional primers, with fewer than 41 additional primers, with fewer than 42 additional primers, with fewer than 43 additional primers, with fewer than 44 additional primers, with fewer than 45 additional primers, with fewer than 46 additional primers, with fewer than 47 additional primers, with fewer than 48 additional primers, with fewer than 49 additional primers, with fewer than 50 additional primers, with fewer than 51 additional primers, with fewer than 52 additional primers, with fewer than 53 additional primers, with fewer than 54 additional primers, with fewer than 55 additional primers, with fewer than 56 additional primers, with fewer than 57 additional primers, with fewer than 58 additional primers, with fewer than 59 additional primers, with fewer than 60 additional primers, with fewer than 61 additional primers, with fewer than 62 additional primers, with fewer than 63 additional primers, with fewer than 64 additional primers, with fewer than 65 additional primers, with fewer than 66 additional primers, with fewer than 67 additional primers, with fewer than 68 additional primers, with fewer than 69 additional primers, with fewer than 70 additional primers, with fewer than 71 additional primers, with fewer than 72 additional primers, with fewer than 73 additional primers, with fewer than 74 additional primers, with fewer than 75 additional primers, with fewer than 76 additional primers, with fewer than 77 additional primers, with fewer than 78 additional primers, with fewer than 79 additional primers, with fewer than 80 additional primers, with fewer than 81 additional primers, with fewer than 82 additional primers, with fewer than 83 additional primers, with fewer than 84 additional primers, with fewer than 85 additional primers, with fewer than 86 additional primers, with fewer than 87 additional primers, with fewer than 88 additional primers, with fewer than 89 additional primers, with fewer than 90 additional primers, with fewer than 91 additional primers, with fewer than 92 additional primers, with fewer than 93 additional primers, with fewer than 94 additional primers, with fewer than 95 additional primers, with fewer than 96 additional primers, with fewer than 97 additional primers, with fewer than 98 additional primers, with fewer than 99 additional primers, with fewer than 100 additional primers, with fewer than 110 additional primers, with fewer than 120 additional primers, with fewer than 130 additional primers, with fewer than 140 additional primers, with fewer than 150 additional primers, with fewer than 160 additional primers, with fewer than 170 additional primers, with fewer than 180 additional primers, with fewer than 190 additional primers, with fewer than 200 additional primers, with fewer than 210 additional primers, with fewer than 220 additional primers, with fewer than 230 additional primers, with fewer than 240 additional primers, with fewer than 250 additional primers, with fewer than 260 additional primers, with fewer than 270 additional primers, with fewer than 280 additional primers, with fewer than 290 additional primers, with fewer than 300 additional primers, with fewer than 310 additional primers, with fewer than 320 additional primers, with fewer than 330 additional primers, with fewer than 340 additional primers, with fewer than 350 additional primers, with fewer than 360 additional primers, with fewer than 370 additional primers, with fewer than 380 additional primers, with fewer than 390 additional primers, with fewer than 400 additional primers, with fewer than 410 additional primers, with fewer than 420 additional primers, with fewer than 430 additional primers, with fewer than 440 additional primers, with fewer than 450 additional primers, with fewer than 460 additional primers, with fewer than 470 additional primers, with fewer than 480 additional primers, with fewer than 490 additional primers, with fewer than 500 additional primers, with fewer than 550 additional primers, with fewer than 600 additional primers, with fewer than 650 additional primers, with fewer than 700 additional primers, with fewer than 750 additional primers, with fewer than 800 additional primers, with fewer than 850 additional primers, with fewer than 900 additional primers, with fewer than 950 additional primers, with fewer than 1,000 additional primers, with fewer than 1,100 additional primers, with fewer than 1,200 additional primers, with fewer than 1,300 additional primers, with fewer than fewer than 1,400 additional primers, with fewer than 1,500 additional primers, with fewer than 1,600 additional primers, with fewer than 1,700 additional primers, with fewer than 1,800 additional primers, with fewer than 1,900 additional primers, with fewer than 2,000 additional primers, with fewer than 2,100 additional primers, with fewer than 2,200 additional primers, with fewer than 2,300 additional primers, with fewer than 2,400 additional primers, with fewer than 2,500 additional primers, with fewer than 2,600 additional primers, with fewer than 2,700 additional primers, with fewer than 2,800 additional primers, with fewer than 2,900 additional primers, with fewer than 3,000 additional primers, with fewer than 3,500 additional primers, or with fewer than 4,000 additional primers.

The amplification reaction can be performed, for example, with fewer than 2 primers, with fewer than 3 primers, with fewer than 4 primers, with fewer than 5 primers, with fewer than 6 primers, with fewer than 7 primers, with fewer than 8 primers, with fewer than 9 primers, with fewer than 10 primers, with fewer than 11 primers, with fewer than 12 primers, with fewer than 13 primers, with fewer than 14 primers, with fewer than 15 primers, with fewer than 16 primers, with fewer than 17 primers, with fewer than 18 primers, with fewer than 19 primers, with fewer than 20 primers, with fewer than 21 primers, with fewer than 22 primers, with fewer than 23 primers, with fewer than 24 primers, with fewer than 25 primers, with fewer than 26 primers, with fewer than 27 primers, with fewer than 28 primers, with fewer than 29 primers, with fewer than 30 primers, with fewer than 31 primers, with fewer than 32 primers, with fewer than 33 primers, with fewer than 34 primers, with fewer than 35 primers, with fewer than 36 primers, with fewer than 37 primers, with fewer than 38 primers, with fewer than 39 primers, with fewer than 40 primers, with fewer than 41 primers, with fewer than 42 primers, with fewer than 43 primers, with fewer than 44 primers, with fewer than 45 primers, with fewer than 46 primers, with fewer than 47 primers, with fewer than 48 primers, with fewer than 49 primers, with fewer than 50 primers, with fewer than 51 primers, with fewer than 52 primers, with fewer than 53 primers, with fewer than 54 primers, with fewer than 55 primers, with fewer than 56 primers, with fewer than 57 primers, with fewer than 58 primers, with fewer than 59 primers, with fewer than 60 primers, with fewer than 61 primers, with fewer than 62 primers, with fewer than 63 primers, with fewer than 64 primers, with fewer than 65 primers, with fewer than 66 primers, with fewer than 67 primers, with fewer than 68 primers, with fewer than 69 primers, with fewer than 70 primers, with fewer than 71 primers, with fewer than 72 primers, with fewer than 73 primers, with fewer than 74 primers, with fewer than 75 primers, with fewer than 76 primers, with fewer than 77 primers, with fewer than 78 primers, with fewer than 79 primers, with fewer than 80 primers, with fewer than 81 primers, with fewer than 82 primers, with fewer than 83 primers, with fewer than 84 primers, with fewer than 85 primers, with fewer than 86 primers, with fewer than 87 primers, with fewer than 88 primers, with fewer than 89 primers, with fewer than 90 primers, with fewer than 91 primers, with fewer than 92 primers, with fewer than 93 primers, with fewer than 94 primers, with fewer than 95 primers, with fewer than 96 primers, with fewer than 97 primers, with fewer than 98 primers, with fewer than 99 primers, with fewer than 100 primers, with fewer than 110 primers, with fewer than 120 primers, with fewer than 130 primers, with fewer than 140 primers, with fewer than 150 primers, with fewer than 160 primers, with fewer than 170 primers, with fewer than 180 primers, with fewer than 190 primers, with fewer than 200 primers, with fewer than 210 primers, with fewer than 220 primers, with fewer than 230 primers, with fewer than 240 primers, with fewer than 250 primers, with fewer than 260 primers, with fewer than 270 primers, with fewer than 280 primers, with fewer than 290 primers, with fewer than 300 primers, with fewer than 310 primers, with fewer than 320 primers, with fewer than 330 primers, with fewer than 340 primers, with fewer than 350 primers, with fewer than 360 primers, with fewer than 370 primers, with fewer than 380 primers, with fewer than 390 primers, with fewer than 400 primers, with fewer than 410 primers, with fewer than 420 primers, with fewer than 430 primers, with fewer than 440 primers, with fewer than 450 primers, with fewer than 460 primers, with fewer than 470 primers, with fewer than 480 primers, with fewer than 490 primers, with fewer than 500 primers, with fewer than 550 primers, with fewer than 600 primers, with fewer than 650 primers, with fewer than 700 primers, with fewer than 750 primers, with fewer than 800 primers, with fewer than 850 primers, with fewer than 900 primers, with fewer than 950 primers, with fewer than 1,000 primers, with fewer than 1,100 primers, with fewer than 1,200 primers, with fewer than 1,300 primers, with fewer than fewer than 1,400 primers, with fewer than 1,500 primers, with fewer than 1,600 primers, with fewer than 1,700 primers, with fewer than 1,800 primers, with fewer than 1,900 primers, with fewer than 2,000 primers, with fewer than 2,100 primers, with fewer than 2,200 primers, with fewer than 2,300 primers, with fewer than 2,400 primers, with fewer than 2,500 primers, with fewer than 2,600 primers, with fewer than 2,700 primers, with fewer than 2,800 primers, with fewer than 2,900 primers, with fewer than 3,000 primers, with fewer than 3,500 primers, or with fewer than 4,000 primers.

The disclosed primers can have one or more modified nucleotides. Such primers are referred to herein as modified primers. Chimeric primers can also be used. Chimeric primers are primers having at least two types of nucleotides, such as both deoxyribonucleotides and ribonucleotides, ribonucleotides and modified nucleotides, two or more types of modified nucleotides, deoxyribonucleotides and two or more different types of modified nucleotides, ribonucleotides and two or more different types of modified nucleotides, or deoxyribonucleotides, ribonucleotides and two or more different types of modified nucleotides. One form of chimeric primer is peptide nucleic acid/nucleic acid primers. For example, 5′-PNA-DNA-3′ or 5′-PNA-RNA-3′ primers may be used for more efficient strand invasion and polymerization invasion. Other forms of chimeric primers are, for example, 5′-(2′-O-Methyl) RNA-RNA-3′ or 5′-(2′-O-Methyl) RNA-DNA-3′.

Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. A primer having one or more universal bases is not considered to be a primer having a specific sequence.

Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)nON[CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoogsteen manner, but which are linked together through a moiety other than a phosphate moiety. Nucleotide substitutes are able to conform to a double helix type structure when interacting with the appropriate nucleic acid molecules.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Primers can be comprised of nucleotides and can be made up of different types of nucleotides or the same type of nucleotides. For example, one or more of the nucleotides in a primer can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can be comprised of bases (that is, the base portion of the nucleotide) and can (and normally will) comprise different types of bases.

### Capture Sequence Design

The capture sequence, also referred to herein as the “second nucleic acid sequence,” is complementary to the template such that it hybridizes to the target nucleic acid molecule downstream from the 3′ end of the primer. In some embodiments, resistance to mutations in the target nucleic acid is desired and the capture sequence is designed with a melting temperature greater than the reaction temperature. In these embodiments, the capture sequence is designed with an isolated Tm of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees above the reaction temperature. For example, the capture, or second, sequence is between about 0° C. and 40° C., between about 5° C. and 30° C., between about 7° C. and 25° C. above the reaction temperature. In some embodiments, the predicted melting temperature of the capture sequence is also made for expected mutants. In these embodiments, the isolated Tm of the capture sequence to the expected mutants is between about 1, 2, 3, 4, 5, 6, 7, 8, 9, 10, 11, 12, 13, 14, 15 16, 17, 18, 19, 20, or more degrees C. below the reaction temperature, or 10, 11, 12, 13, 14,1 5, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 or more degrees C. above the reaction temperature. For example, it can be 10° C. below the reaction temperature and 30° C. above the reaction temperature, between about 3° C. below the reaction temperature and about 10° C. above the reaction temperature.

To achieve these melting temperatures, the capture sequence length can be 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 or more bases in length. For example, it can be between about 20 and about 50, between about 22 and about 40, between about 23 and about 37 bases.

In some embodiments, an even higher resistance to mutations in the target sequence is desired. In these embodiments, in addition to a capture sequence with an isolated Tm of between about 0° C. and 40° C. above the reaction temperature, the cooperative primer is designed with an isolated Tm of between about 7° C. below and about 20° C. above, between about 5° C. below and about 10° C. above, between about 3° C. below and about 3° C. above the reaction temperature. The cooperative interaction between the primer and the capture sequence will result in an even greater effective Tm for the cooperative primer, rendering it almost impervious to mutations in the sequence. By comparison, a normal primer might have to be an additional 5 to 30 bases in length to have an equivalent resistance to mutations in the target sequence, and consequently, would be much more susceptible to primer-dimer formation.

In other embodiments, a higher resistance to primer-dimers is preferred and the melting temperature of the isolated capture, or second, nucleic acid sequence is designed to be less than the reaction temperature. For example, the capture, or second, nucleic acid sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more degrees below the reaction temperature, or annealing phase, of PCR .In preferred embodiments, the Tm of the isolated capture, or second nucleic acid, sequence is between about 0° C. and 12° C., between about 1° C. and 8° C., between about 2° C. and 5° C. below the reaction temperature. To achieve these low melting temperatures, the capture sequence length can be 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more bases in length. For example, the capture, or second nucleic acid sequence, can be between about 5 and 30, between about 8 and 25, and between about 10 and 22 bases.

In some embodiments, the capture sequence binds and releases the target sequence rapidly such that the polymerase can extend underneath the capture sequence, leaving the capture sequence intact. In some embodiments, this is enhanced using a cooperative primer with the linker attached to the 5′ end of the capture sequence. In a preferred embodiment, the polymerase is capable of cleaving the capture sequence during extension. In a preferred embodiment, this is enhanced using a cooperative primer with the linker attached to the 3′ end of the capture sequence.

### Linker

The number of bases between the 3′ end of the first nucleic acid, or primer, sequence and the 5′ end of the second nucleic acid, or capture sequence hybridization locations in the template is important. In some embodiments, the number of bases between the primer and the capture sequence is 1, 2, 3, 4, 5, 6, 7, 8, 9, 10, 11, 12, 13, 14, 15, 16,17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. For example, they can be between about 0 and 30, between about 0 and 20, between about 0 and 10 bases.

The more bases that are between the two sites, the longer the linker needs to be if cleavage of the capture sequence is desired. The longer the linker, the more entropy that enters into the system, which lowers the effect of cooperative binding. This is expressed in the following equation:

K_{eff}=Kₚᵣᵢₘₑᵣ+K_{capture}+L_{C}KₚᵣᵢₘₑᵣK_{capture}

Where Keff is the effective or cooperative equilibrium constant, Kprimer is the equilibrium constant of the primer in isolation, Kcapture is the equilibrium constant of the capture sequence in equilibrium and Lc is the local concentration defined as:

$${L}_{c} = \frac{( \frac{1}{6.022 E 23} )}{\frac{4}{3} π {r}^{3}}$$

Where r is the linker length in decimeters. This provides the effective local concentration in molarity due to the cooperative interaction between the primer and the probe. Accordingly, linker length directly determines the cooperative contribution (L_{c}KₚᵣᵢₘₑᵣK_{capture}) to the effective equilibrium constant.

Kₚᵣᵢₘₑᵣ and K_{capture} can be calculated by obtaining the enthalpy and entropy values for the primer and the capture sequences using nearest neighbor or other calculations known to those skilled in the art.

The total amount of template bound by the primer can be calculated as follows:

$$\frac{{T}_{primer}}{{T}_{o}} = \frac{( {K}_{primer} + {L}_{C} {K}_{primer} {K}_{capture} ) {P}_{o}}{1 + ( {K}_{primer} + {K}_{capture} + {L}_{C} {K}_{primer} {K}_{capture} ) {P}_{o}}$$

Where Tprimer is the template bound by primer, Tₒ is the total amount of template and Pₒ is the starting cooperative primer concentration. It can be seen that the cooperative effect is greatest when L_{c}KₚᵣᵢₘₑᵣK_{capture} is much greater than Kₚᵣᵢₘₑᵣ. For this to occur the linker length should be as short as possible.

While the math shows that the linker length should be as short as possible, there are several limitations to how short the linker can actually be. When the capture sequence and the probe bind to the template, they form rigid double helices. The linker length must be sufficient to accommodate this structure.

In some embodiments, the linker attaches the 5′ end of the primer to the 3′ end of the capture sequence. In this embodiment, the linker is larger than the combined length of the primer and capture sequences. In a preferred embodiment where the linker attaches to the 3′ end of the capture sequence, the linker comprises 6 hexaethylene glycols. In another embodiment, the primer is inverted such that the 5′ end of the primer is attached to the 5′ end of the capture sequence. In this embodiment, the linker is longer than the primer. In a preferred embodiment where the linker attaches to the 5′ end of the capture sequence, the linker comprises 3 hexaethylene glycols. In yet another embodiment, the 3′ end of the capture sequence is linked to the middle of the primer. In this instance, the linker may be shorter than the length of the primer.

A variety of linker types and compositions are known to those skilled in the art. Examples include, but are not limited to, polyethylene glycol and carbon linkers. Linkers can be attached through a variety of methods, including but not limited to, covalent bonds, ionic bonds, hydrogen bonding, polar association, magnetic association, and van der wals association. A preferred method is covalent bonding through standard DNA synthesis methods.

The length of polyethylene glycol linkers is about 0.34 nm per monomer. In some embodiments, the length of the polyethylene glycol linker is between about 1 and 90, between about 2 and 50, between about 3 and 30 monomers (between about 1 and 10 nm fully extended).

### Using a Primer with a Built in Detection Mechanism

In some embodiments, the primer has a built in detection mechanism. In some embodiments the detection mechanism includes one or more detectable labels. In a preferred embodiment, the detection mechanism includes a FRET pair. Examples of primers with built in detection mechanisms include, but are not limited to, Amplifluor primers, Rapid Detex primers, and others known to those skilled in the art.

Cooperative nucleic acids with built in detection mechanisms can be more useful to assay designers than non-cooperative nucleic acids (normal primers) with built in detection mechanisms. Without being limited by theory, this is because cooperative nucleic acids are less prone to generate signal from nonspecific products, such as primer-dimers.

In some embodiments, a nucleic acid binding dye, such as SYBR Green, is used to monitor the progress of the amplification reaction.

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers. The use of several types of fluorescent change probes and primers are reviewed in Schweitzer and Kingsmore, Curr. Opin. Biotech. 12:21-27 (2001). Hall et al., Proc. Natl. Acad. Sci. USA 97:8272-8277 (2000), describe the use of fluorescent change probes with Invader assays.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes (Holland et al., Proc. Natl. Acad. Sci. USA 88:7276-7280 (1991)) are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers (Nazerenko et al., Nucleic Acids Res. 25:2516-2521 (1997)) and scorpion primers (Thelwell et al., Nucleic Acids Res. 28(19):3752-3761 (2000)).

Cleavage activated primers are similar to cleavage activated probes except that they are primers that are incorporated into replicated strands and are then subsequently cleaved. Little et al., Clin. Chem. 45:777-784 (1999), describe the use of cleavage activated primers.

Multiplexing with ARMS

In some embodiments, detection of multiple polymorphisms, insertions, deletions or other mutations is desired. In some embodiments, the primer is designed such that the base on the 3′ end is over the mutation. In some embodiments, additional intentional polymorphisms are designed into the primer. In one embodiment, the presence of a probe attached to the primer allows for allele specific real-time detection of multiple polymorphisms in the same location.

Mutation Differentiation with the Probe

In some embodiments the differentiation of polymorphisms is accomplished using the capture sequence attached to the primer. In some embodiments the capture sequence has additional mutations intentionally added to improve differentiation. In some embodiments, the capture sequence will not bind when a polymorphism is present, preventing efficient amplification round after around. In some embodiments where the capture sequence has a detectable label, even if some amplification does occur, the capture sequence does not bind sufficiently to generate a detectable signal.

### Oligonucleotide Synthesis

Primers, detection probes, address probes, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the intemucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. Nos. 6,294,664 and 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor, or a mixture of one or more different nucleotide precursors. For the disclosed primers of specific sequence, specific nucleotide precursors would be added sequentially.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

So long as their relevant function is maintained, primers, detection probes, address probes, and any other oligonucleotides can be made up of or include modified nucleotides (nucleotide analogs). Many modified nucleotides are known and can be used in oligonucleotides, and are disclosed elsewhere herein.

### Kits

The materials described above as well as other materials can be packaged together in any suitable combination as a kit useful for performing, or aiding in the performance of, the disclosed method. It is useful if the kit components in a given kit are designed and adapted for use together in the disclosed method. For example Also disclosed herein is a kit, wherein the kit comprises a cooperative nucleic acid primer (“co-primer”), wherein said co-primer comprises: a first nucleic acid sequence, wherein the first nucleic acid sequence is complementary to a first region of the target RNA, and wherein the first nucleic acid sequence is extendable on the 3′ end; a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence; a linker connecting said first and second nucleic acid sequences in a manner that allows both the said first and second nucleic acid sequences to hybridize to the target RNA at the same time; a conditionally active reverse transcriptase primer, wherein said conditionally active reverse transcriptase primer has a melting temperature between about 45° C. and about 55° C. The kit can further comprise reverse transcriptase.

### Detection

Products of amplification can be detected using any nucleic acid detection technique. For real-time detection, the amplification products and the progress of amplification are detected during amplification. Real-time detection is usefully accomplished using one or more or one or a combination of fluorescent change probes and fluorescent change primers. Other detection techniques can be used, either alone or in combination with real-timer detection and/or detection involving fluorescent change probes and primers. Many techniques are known for detecting nucleic acids. The nucleotide sequence of the amplified sequences also can be determined using any suitable technique.

### EXAMPLES

### Example I: Reverse Transcription Using Co-Primers

### Materials and Methods

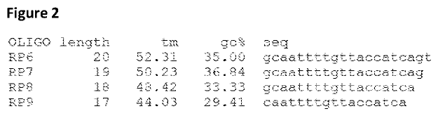

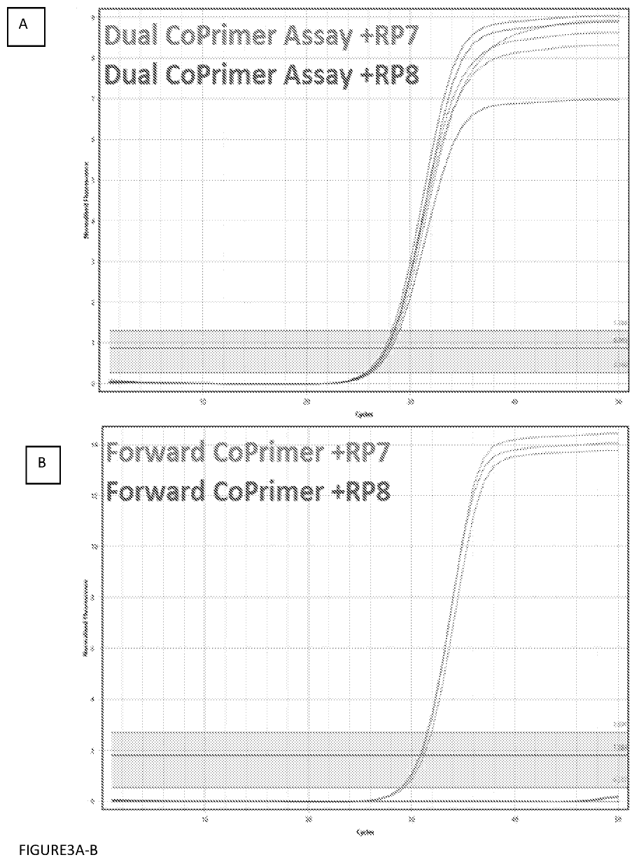

Candidate RT Primers were designed for SARS-CoV-2 Rdrp, 50 to 300 bp downstream of the associated Reverse CoPrimer. Candidates were analyzed for in silico inclusivity and exclusivity equivalent to the CoPrimer assay. Candidates were designed with a variety of lengths and positions, to yield a range of predicted annealing temperatures, between 44° C. and 52° C. Selecting a range of predicted annealing temperatures (FIG. 2) allowed empirical evaluation of PCR amplification competency under standard reaction conditions (FIG. 3).

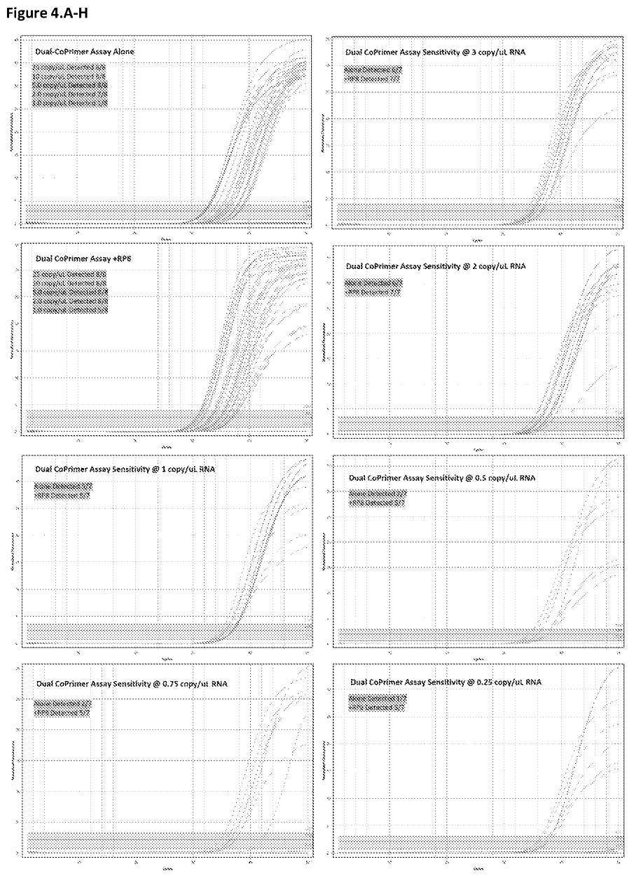

The impact of PCR incompetent RP8 on Dual CoPrimer assay sensitivity was then evaluated by Detection Rate for a range of RNA concentrations near the Limit of Detection (LoD) of the assay (FIG. 4).

As depicted, we found that including RP8 consistently improved the Detection Rate of the Dual CoPrimer assay across numerous near-LoD levels, in paired reactions in the same run with otherwise identical chemistry and cycling conditions. Based on probit calculation, this improved detection rate corresponds to approximately 2.3-fold improvement in sensitivity.

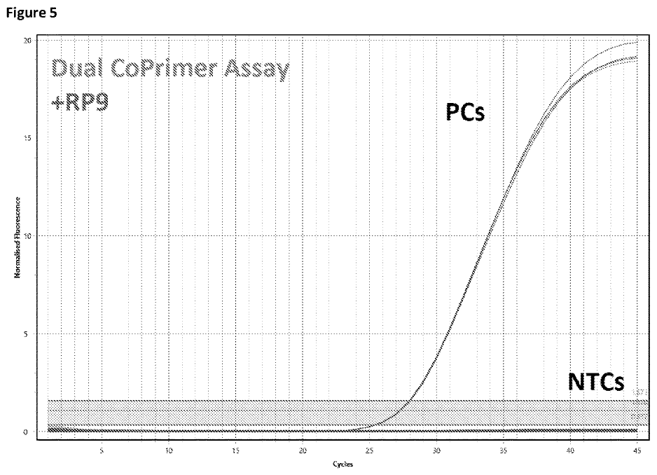

To determine whether including a target-specific RT primer would increase reaction complexity and thereby increase the rate of non-specific amplification, four PC reactions and 44 replicate reactions of No-Template Controls were performed with another RT Primer (RP9) included (FIG. 5). To exacerbate any potential NTC amplification, the complete reactions were incubated at Room Temperature for an additional 10 minutes prior to RT-PCR cycling. As depicted, both the Dual CoPrimer assay and the Dual CoPrimer assay with RP9 retained full specificity in NTC reactions under these exacerbated experimental conditions.

These findings support the utility of the disclosed empirically selected, conditionally active RT primers to improve the sensitivity of CoPrimer assays for RNA targets without diminished specificity.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are specifically incorporated by reference. Nothing herein is to be construed as an admission that the present invention is not entitled to antedate such disclosure by virtue of prior invention.

Those skilled in the art will recognize, or be able to ascertain using no more than routine experimentation, many equivalents to the specific embodiments of the method and compositions described herein. Such equivalents are intended to be encompassed by the following claims.

1. A method of transcribing RNA into cDNA, the method comprising:
a. providing a target RNA sequence;
b. providing a cooperative nucleic acid primer (“co-primer”), wherein said co-primer comprises:
i. a first nucleic acid sequence, wherein the first nucleic acid sequence is complementary to a first region of the target RNA, and wherein the first nucleic acid sequence is extendable on the 3′ end;
ii. a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence;
iii. a linker connecting said first and second nucleic acid sequences in a manner that allows both the said first and second nucleic acid sequences to hybridize to the target RNA at the same time;
c. providing a conditionally active reverse transcriptase primer, wherein said conditionally active reverse transcriptase primer has a melting temperature between about 45° C. and about 55° C.;
d. providing reverse transcriptase; and
e. providing conditions that allow for transcription of the RNA target into cDNA;
thereby transcribing RNA into cDNA.

2. The method of claim 1, wherein the conditionally active reverse transcriptase primer binds the RNA target at a distance of at least 5 nucleotides from where the co-primer binds the RNA target.

3. The method of claim 2, wherein the conditionally active reverse transcriptase primer binds the RNA target at a distance of at least 10 nucleotides from where the co-primer binds the RNA target.

4. The method of claim 3, wherein the conditionally active reverse transcriptase primer binds the RNA target at a distance of at least 50 nucleotides, but no more than 300 nucleotides, from where the co-primer binds the RNA target.

5. The method of claim 1, wherein the conditionally active reverse transcriptase primer binds the RNA target at a distance between about 10 and 200 nucleotides from where the co-primer binds the RNA target.

6. The method of claim 1, wherein the first nucleic acid molecule of the co-primer will not hybridize to the target without the second nucleic acid molecule hybridizing to the target.

7. The method of claim 1, wherein the second nucleic acid molecule of the co-primer will not hybridize to the target without the first nucleic acid molecule hybridizing to the target.

8. The method of claim 1, wherein neither the first nor the second nucleic acid molecule of the co-primer will hybridize to the target without the other hybridizing to the target.

9. A kit comprising:
a. a cooperative nucleic acid primer (“co-primer”), wherein said co-primer comprises:
i. a first nucleic acid sequence, wherein the first nucleic acid sequence is complementary to a first region of the target RNA, and wherein the first nucleic acid sequence is extendable on the 3′ end;
ii. a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence;
iii. a linker connecting said first and second nucleic acid sequences in a manner that allows both the said first and second nucleic acid sequences to hybridize to the target RNA at the same time;
b. a conditionally active reverse transcriptase primer, wherein said conditionally active reverse transcriptase primer has a melting temperature between about 45° C. and about 55° C.

10. The kit of claim 9, wherein said kit further comprises reverse transcriptase.

11. A method of detecting RNA, the method comprising:
a. providing a target RNA sequence;
b. providing a cooperative nucleic acid primer (“co-primer”), wherein said co-primer comprises:
i. a first nucleic acid sequence, wherein the first nucleic acid sequence is complementary to a first region of the target RNA, and wherein the first nucleic acid sequence is extendable on the 3′ end;
ii. a second nucleic acid sequence, wherein the second nucleic acid sequence is complementary to a second region of the target RNA, such that in the presence of the target RNA, the second nucleic acid sequence hybridizes to the target RNA downstream from the 3′ end of the first nucleic acid sequence;
iii. a linker connecting said first and second nucleic acid sequences in a manner that allows both the said first and second nucleic acid sequences to hybridize to the target RNA at the same time;
c. providing a conditionally active reverse transcriptase primer, wherein said conditionally active reverse transcriptase primer has a melting temperature between about 45° C. and about 55° C.;
d. providing reverse transcriptase;
e. providing conditions that allow for transcription of the RNA target into cDNA, thereby producing cDNA;
f. amplifying the cDNA product of step e); and
g. detecting the amplified cDNA produced in step f.

12. The method of claim 11, wherein said RNA is a virus.

13. The method of claim 12, wherein said virus is SARS-CoV-2.

14. The method of claim 11, wherein said amplification comprises polymerase chain reaction (PCR).

15. The method of claim 11, wherein co-primers are used in the amplification of step f).

16. The method of claim 15, wherein at least one co-primer used in amplification is different than the co-primer used in reverse transcription.

17. The method of claim 11, wherein said amplification comprises isothermal amplification.

18. The method of step 11, wherein before said amplification step, appropriate reagents and conditions for amplification are provided.

19. The method of claim 11, wherein the conditionally active reverse transcriptase primer binds the RNA target at a distance of at least 5 nucleotides from where the co-primer binds the RNA target.

20. The method of claim 19, wherein the conditionally active reverse transcriptase primer binds the RNA target at a distance of at least 10 nucleotides from where the co-primer binds the RNA target.

21. The method of claim 20, wherein the conditionally active reverse transcriptase primer binds the RNA target at a distance of at least 10 nucleotides, but no more than 300 nucleotides, from where the co-primer binds the RNA target.

22. The method of claim 11, wherein the conditionally active reverse transcriptase primer binds the RNA target at a distance between about 10 and 200 nucleotides from where the co-primer binds the RNA target.

23. The method of claim 11, wherein the first nucleic acid molecule of the co-primer will not hybridize to the target without the second nucleic acid molecule hybridizing to the target.

24. The method of claim 11, wherein the second nucleic acid molecule of the co-primer will not hybridize to the target without the first nucleic acid molecule hybridizing to the target.

25. The method of claim 11, wherein neither the first nor the second nucleic acid molecule of the co-primer will hybridize to the target without the other hybridizing to the target.


SEQUENCE LISTING (4 sequences)

SEQ ID NO: 1 - DNA, 20 bp, Artificial Sequence
  SYNTHETIC CONSTRUCT
gcaattttgttaccatcagt

SEQ ID NO: 2 - DNA, 19 bp, Artificial Sequence
  SYNTHETIC CONSTRUCT
gcaattttgttaccatcag

SEQ ID NO: 3 - DNA, 18 bp, Artificial Sequence
  SYNTHETIC CONSTRUCT
gcaattttgttaccatca

SEQ ID NO: 4 - DNA, 17 bp, Artificial Sequence
  SYNTHETIC CONSTRUCT
caattttgttaccatca